(19) United States
(12) Patent Application Publication
(10) Pub. No.: US 2013/0345775 A1
(43) Pub. Date: Dec. 26, 2013

(54) Determining Control Settings for a Hearing Prosthesis

(71) Applicants: Bjorn Davidsson, Gothenburg (SE); Edin Krijestorac, Gothenburg (SE); Mark Christopher Flynn, Gothenburg (SE)

(72) Inventors: Bjorn Davidsson, Gothenburg (SE); Edin Krijestorac, Gothenburg (SE); Mark Christopher Flynn, Gothenburg (SE)

(73) Assignee: COCHLEAR LIMITED, Macquarie University (AU)

(21) Appl. No.: 13/530,066

(22) Filed: Jun. 21, 2012

(51) Int. Cl. A61F 11/04; H04R 25/00

(52) U.S. Cl. 607/57

(57) ABSTRACT

Methods, systems, and devices for determining control settings used by a hearing prosthesis to process a sound are disclosed. A first model output based on control settings is received by a computing device configured to fit the hearing prosthesis to a user. A difference between the first model output and a reference output based on normal human hearing at a target frequency is determined. If the difference between the first model output and the reference output at the target frequency is within a specification, the computing device sends a signal to the hearing prosthesis that includes information indicative of the control settings.

### BACKGROUND

Due to hearing loss, some individuals have difficulty perceiving or are unable to perceive sound. In order to perceive a least a portion of a sound, individuals with hearing loss may benefit from the use of a hearing prosthesis. Certain hearing prostheses are designed to assist users having specific types of hearing loss. The effectiveness of a hearing prosthesis depends on the type and severity of a user's hearing loss. Furthermore, depending on the hearing prosthesis, the user may perceive sound as a person with normal hearing, or the hearing prosthesis may allow the user to perceive a portion of the sound.

The effectiveness of the hearing prosthesis also depends on how well the prosthesis is configured for, or “fitted” to, a user of the hearing prosthesis. Fitting the hearing prosthesis, sometimes also referred to as “programming,” “calibrating,” or “mapping,” creates a set of control settings and other data that define the specific characteristics of the stimuli (in the form of acoustic, mechanical, or electrical signals) delivered to the relevant portions of the person's outer ear, middle ear, inner ear, or auditory nerve. The control settings are based on each individual user's type and severity of hearing loss. This configuration information is sometimes referred to as the user's “program” or “MAP.”

### SUMMARY

A first method for determining control settings for a hearing prosthesis is disclosed. The first method includes receiving, by a computing device configured to fit a hearing prosthesis to a user of the hearing prosthesis, a model output of the hearing prosthesis. The model output is based on control settings usable by the hearing prosthesis to process a sound. The first method also includes determining if the model output passes a validation test. The validation test includes determining a difference between the model output and a reference output at a target frequency. The reference output is based on normal human hearing. The validation test also includes determining whether the difference between the model output and the reference output at the target frequency is within a specification. In response to determining that the model output passes the validation test, the first method includes sending a signal to the hearing prosthesis that contains information indicative of the control settings.

A second method for determining control settings of a hearing prosthesis is also disclosed. The second method includes receiving, by a computing device configured to fit a hearing prosthesis to a user of the hearing prosthesis, a first model output of the hearing prosthesis, a second model output of the hearing prosthesis, and a reference output. The first model output is based on first control settings usable by the hearing prosthesis to process a sound. The second model output is based on second control settings usable by the hearing prosthesis to process the sound. The reference output is based on normal human hearing. The second method also includes determining a first weighted difference between the first model output and the reference output, which includes a first difference at a first frequency and a second difference at a second frequency. The first difference is given more weight than the second difference. The second method also includes determining a second weighted difference between the second model output and the reference output that includes a third difference at the first frequency and a fourth difference at the second frequency. The third difference is given more weight than the fourth difference. The second method additionally includes determining that the first weighted difference is less than the second weighted difference. The second method further includes determining that first weighted difference is within a tolerance. In response to determining that the first weighted difference is within the tolerance, the second method includes sending a signal to the hearing prosthesis that includes information indicative of the first control settings.

A device is also disclosed. The device includes an interface component configured to send one or more control settings to a hearing prosthesis that are usable by the hearing prosthesis to process a sound. The device also includes a processor. The processor is configured to receive a first model output of the hearing prosthesis based on initial control settings. The processor is also configured to determine whether a first output characteristic of the first model output is outside of a specification by determining whether a first output characteristic of the first model output at a target frequency exceeds a threshold value. In response to determining that the first output characteristic is outside of the specification, the processor is configured to generate second control settings and send a signal to the hearing prosthesis that includes information indicative of the second control settings. The second control settings results in a second model output having second output characteristic that is within the specification.

These as well as other aspects and advantages will become apparent to those of ordinary skill in the art by reading the following detailed description, with reference where appropriate to the accompanying drawings. Further, it is understood that this summary is merely an example and is not intended to limit the scope of the invention as claimed.

### BRIEF DESCRIPTION OF THE FIGURES

Presently preferred embodiments are described below in conjunction with the appended drawing figures, wherein like reference numerals refer to like elements in the various figures, and wherein:

FIG. 1 is a block diagram of a fitting system, according to an example;

FIG. 2 is a block diagram of a hearing prosthesis depicted in FIG. 1, according to an example;

FIG. 3 is a block diagram of a computing device depicted in FIG. 1, according to an example;

FIG. 4 is a flow diagram of a method for determining control settings of a hearing prosthesis, according to an example;

FIG. 5 is a flow diagram of a first method for iteratively determining control settings of a hearing prosthesis, according to an example;

FIG. 6 is a flow diagram of a method for validating a model output, according to an example;

FIG. 7 is a flow diagram of a method for validating a target frequency, according to an example; and

FIG. 8 is a flow diagram of a second method for iteratively determining control signals for a hearing prosthesis, according to an example.

### DETAILED DESCRIPTION

The following detailed description describes various features, functions, and attributes of the disclosed systems, methods, and devices with reference to the accompanying figures. In the figures, similar symbols typically identify similar components, unless context dictates otherwise. The illustrative embodiments described herein are not meant to be limiting. Certain aspects of the disclosed systems, methods, and devices can be arranged and combined in a wide variety of different configurations, all of which are contemplated herein.

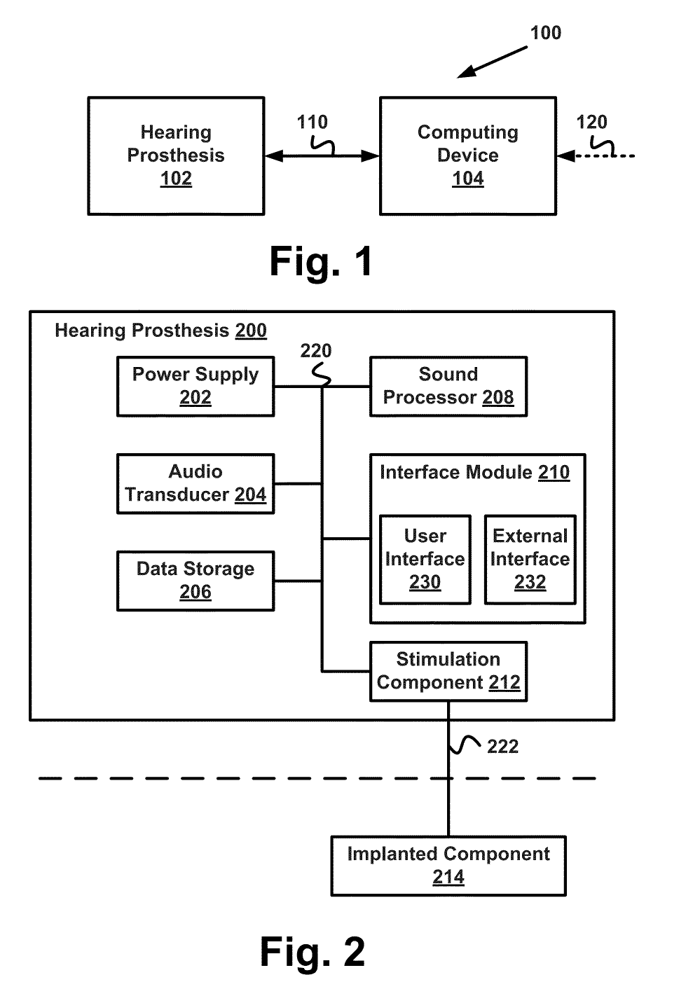

FIG. 1 is a block diagram of a fitting system 100. The fitting system 100 includes a hearing prosthesis 102 and a computing device 104. An audiologist, or similar specialist, uses the fitting system 100 to fit the hearing prosthesis 102 to a user of the hearing prosthesis 102. Alternatively, any person, including the user of the hearing prosthesis 102, may use the fitting system 100 to fit the hearing prosthesis 102 to the user.

In one example, the hearing prosthesis 102 is a bone conduction device. In another example, the hearing prosthesis 102 is a cochlear implant, a direct acoustic stimulation device, a brain stem implant, a middle ear implant, or any other hearing prosthesis or combination of hearing prostheses now known or later discovered that is configured to assist a user of the hearing prosthesis in perceiving at least a portion of a sound.

The audiologist uses the computing device 104 to fit the hearing prosthesis 102 to the user. The computing device 104 is connected to the hearing prosthesis 102 via a link 110. In one example, the link 110 is a wired connection. In another example, the link 110 is a wireless connection.

When performing an initial fit of the hearing prosthesis 102, the computing device 104 receives an input 120 from the audiologist that includes information indicative of the initial control settings for the hearing prosthesis 102. When the audiologist is fine tuning the hearing prosthesis 102 to the user, such as after determining a change in the user's hearing loss, the computing device 104 receives the initial control settings from the hearing prosthesis 102 via the link 110.

Because of the complexity of human hearing, the hearing prosthesis 102 uses a plurality of parameters to process sound. In order to have a workable fitting process, a subset of the parameters is adjusted during the fitting process. Other parameters are fixed and are not adjustable. The control settings determined by the computing device 104 represent a setting for at least one of the parameters included in the subset of parameters. Thus, while the control settings are referred to in the plural, it is understood that the control settings may include one setting for one parameter.

The computing device 104 determines the control settings for the hearing prosthesis 102 using a fitting method. The process by which the computing device 104 determines the control settings the hearing prosthesis 102 is described with respect to FIG. 4.

FIG. 2 is a block diagram of a hearing prosthesis 200. The hearing prosthesis 200 is one example of the hearing prosthesis 102 of the fitting system 100. The hearing prosthesis 200 includes a power supply 202, an audio transducer 204, a data storage 206, a sound processor 208, an interface module 210, and a stimulation component 212, all of which are connected either directly or indirectly via circuitry 220. The hearing prosthesis 200 also includes an implanted component 214 that is connected to the stimulation component 212 via a link 222.

In FIG. 2, the hearing prosthesis 200 is a partially implantable hearing prosthesis, such as a bone conduction device. In this example, the implanted component 214 is implanted in a body of the user of the hearing prosthesis 200, and the components 202-212 of the hearing prosthesis 200 are contained in a single enclosure that the user wears externally on the user's body. Alternatively, the components 202-212 of the hearing prosthesis 200 are contained in one or more connected enclosures that the user wears externally on the user's body. In another example, the hearing prosthesis 200 is a totally implantable hearing prosthesis, such as a totally implantable cochlear implant. In this example, the components 202-214 of the hearing prosthesis 200 are implanted in the user's body in one or more enclosures.

The power supply 202 supplies power to various components of the hearing prosthesis 200 and can be any suitable power supply, such as a rechargeable or a non-rechargeable battery. In one example, the power supply 202 is a battery that can be charged wirelessly, such as through inductive charging. In another example, the power supply 202 is not a replaceable or rechargeable battery and is configured to provide power to the components of the hearing prosthesis 200 for the operational lifespan of the hearing prosthesis 200.

The audio transducer 204 receives a sound from an environment and sends a sound signal to the sound processor 208. In one example, the hearing prosthesis 200 is a bone conduction device, and the audio transducer 204 is an omnidirectional microphone. In another example, the hearing prosthesis 200 is a cochlear implant, an auditory brain stem implant, a direct acoustic stimulation device, a middle ear implant, or any other hearing prosthesis now known or later developed that is suitable for assisting a user of the hearing prosthesis 200 in perceiving sound. In this example, the audio transducer 204 is an omnidirectional microphone, a directional microphone, an electro-mechanical transducer, or any other audio transducer now known or later developed suitable for use in the type of hearing prosthesis employed. Furthermore, in other examples the audio transducer 204 includes one or more additional audio transducers.

The data storage 206 includes any type of non-transitory, tangible, computer readable media now known or later developed configurable to store program code for execution by the hearing prosthesis 200 and/or other data associated with the hearing prosthesis 200. The data storage 206 stores control settings and variable settings usable by the sound processor 208 to process a sound. The data storage 206 may also store computer programs executable by the sound processor 208.

The sound processor 208 receives a sound signal and processes the sound signal into a processed signal suitable for use by the stimulation component 212. In one example, the sound processor 208 is a digital signal processor. In another example, the sound processor 208 is any processor or combination of processors now known or later developed suitable for use in a hearing prosthesis. Additionally, the sound processor 208 may include additional hardware for processing the sound signal, such as analog-to-digital converter.

The sound processor 208 is configured to process a sound having a frequency that is within a frequency range. In one example, the frequency range is from about 250 Hz to about 8 KHz. In another example, the frequency range of the hearing prosthesis is any range of frequencies suitable for allowing the user to perceive at least a portion of a sound.

To process the sound signal, the sound processor 208 accesses the data storage 206 to identify the control settings and the variable settings. In one example, the sound processor 208 also executes a program stored in the data storage 206 to process the sound signal. The sound processor 208 sends the processed signal to the stimulation component 212.

In one example, the control settings include a setting for a parameter used by the sound processor 208 to process a sound. In one example, the parameter includes a gain, a maximum power offset, a compression ratio, a minimum frequency, a maximum frequency, or any other parameter usable by the sound processor 208 to process the sound. In another example, the control settings includes M sets of settings for one or more parameters corresponding to M frequencies, where M is an integer greater than or equal to one. For instance, if there are three frequencies and three parameters, the control settings include the following subsets:
S₁={A₁, B₁, C₁}
S₂={A₂, B₂, C₂}
S₃={A₃, B₃, C₃}
   where S₁₋₃ are sets of parameter settings at a first frequency, a second frequency, a third frequency, respectively; A₁₋₃ are first parameter settings; B₁₋₃ are second parameter settings; and C₁₋₃ are third parameter settings. To adjust the control settings stored in the data storage 206, the hearing prosthesis 200 is fit to the user of the hearing prosthesis 200 using a fitting system, such as the fitting system 100 depicted in FIG. 1.

The variable setting includes a setting for a variable parameter that is adjustable by the user via the interface module 210. In one example, the variable parameter includes a volume, a speech processing mode, or any other parameter suitable for adjustment by the user of the hearing prosthesis 200.

The interface module 210 is configured to receive an input from and/or send an output to one or more external sources. In FIG. 2, the interface module 210 includes a user interface 230 and an external interface 232. The user interface 230 is configured to receive a user input from the user of the hearing prosthesis 200. The external interface 232 is configured to receive an external input from the computing device 104 or another computing device. The external interface 232 is also configured to send the output to the computing device 104. In another example, the interface module 210 may contain more or fewer components configured to receive inputs from and send outputs to one or more additional external sources. The interface module 210 may also include one or more processors.

The user interface 230 receives the user input, which includes information indicative of a user-requested change to a variable setting. The user interface 230 includes a touchpad, a button, a switch, or any component now known or later discovered suitable for receiving the user input from the user of the hearing prosthesis 200. When the user interacts with the user-interface component 230 to change the variable setting, the interface module 210 receives the user input from the user interface component 230. The interface module 210 stores the user-requested change to the variable setting in the data storage 206.

The external interface 232 includes one or more interfaces suitable for connecting the hearing prosthesis 200 to the computing device 104. In one example, the external interface 232 connects the hearing prosthesis 200 to the computing device 104 via a wireless interface. In another example, the external interface 232 connects the hearing prosthesis 200 to the computing device 104 via a wired interface. In yet another example, the external interface 232 is configured to connect the hearing prosthesis 200 to multiple devices. In this example, the external interface includes one or more wireless and/or wired interfaces.

The external interface 232 receives the external input from the computing device 104. In one example, the external input includes information indicative of the control settings. The interface module 210 receives the external input signal from the external interface component 232, and the interface module 210 stores the information indicative of the control settings in the data storage 206.

In another example, the external input includes a request for the control settings. The interface module 210 receives the external input from the external interface 232 and accesses the data storage 206 to identify the control settings. The interface module 210 generates an output signal, which includes information indicative of the control settings. The interface module 210 sends the output signal to the computing device via the external interface 232.

The stimulation component 212 receives the processed signal from the sound processor 208 and generates a stimulation signal based on the processed signal. In an example in which the hearing prosthesis 200 is a bone conduction device, the stimulation component 212 generates the stimulation signal as a mechanical output force in the form of a vibration. In another example, the hearing prosthesis 200 is a cochlear implant, and the stimulation component 212 generates the stimulation signal as an electrical signal capable of activating one or more electrodes of an electrode array implanted in one of the user's cochleae. In yet another example, the stimulation component 212 generates a stimulation signal suitable for use in stimulating a body part of the user of the hearing prosthesis so as to allow the use to perceive a portion of a sound.

The implanted component 214 receives the stimulation signal from the stimulation component 212 via the link 222. In one example, the link 222 is a transcutaneous link. In another example, the link 222 is a percutaneous link.

The implanted component 214 delivers a stimulus to a body part of the user that allows the user to perceive a portion of a sound. In one example, the hearing prosthesis 200 is a bone conduction device, and the implanted component 214 includes an anchor system. The anchor system delivers the stimulus to the user in the form of a vibration applied to a bone in the user's skull. The vibration causes fluid in the user's cochlea to move, thereby activating hair cells in the user's cochlea. The hair cells stimulate an auditory nerve, which allows the user to perceive at least a portion of a sound. In another example, the hearing prosthesis 200 is a cochlear implant, a direct acoustic stimulation device, a brain stem implant, a middle ear implant, or any other hearing prosthesis now known or later discovered. In this example, the stimulus delivered by the implanted component 214 is an electrical stimulus, a mechanical stimulus, or any other stimulus or combination of stimuli capable of stimulating a body part of the user so as to allow the user to perceive at least a portion of a sound.

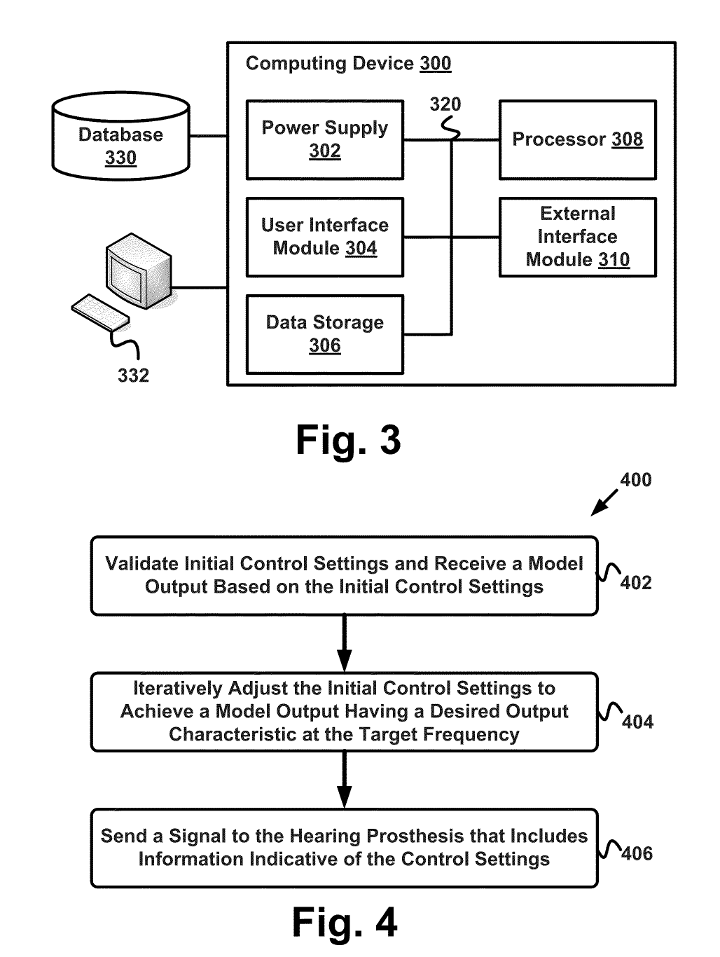

FIG. 3 is a block diagram of a computing device 300. The computing device 300 is one example of the computing device 104 of the fitting system 100. The computing device 300 includes a power supply 302, a user interface module 304, a data storage 306, a processor 308, and an external interface module 310, all of which are connected either directly or indirectly via circuitry 320.

The power supply 302 provides power to components of the computing device 300. In one example, the power supply 302 is connected to a mains power distribution, such as an electrical outlet that supplies 120 VAC power. The power supply 302 includes electrical equipment, such as one or more transformers, that are configured to reduce the power received from the mains power distribution to a voltage suitable for use by the component of the computing device 300. The power supply 302 also includes one or more AC-DC converters. In another example, the power supply 302 includes a rechargeable battery configured to supply power to the components of the computing device 302.

The user interface module 304 is configured to receive an input from a user of the computing device 300 and to provide an output to the user. The user interface module 304 includes at least one input component capable of receiving an input from the user, such as a keyboard, a keypad, a computer mouse, a touch screen, a track ball, a joystick, and/or any other similar device now known or later discovered. The user interface module 304 includes at least one output component capable of displaying information to the user, such as a monitor, touch screen, printer, speaker, and/or any other similar device now known or later discovered.

The data storage 306 includes any type of non-transitory, tangible, computer readable media now known or later developed configurable to store program code for execution by the computing device 300 and/or other data associated with the computing device 300. The data storage 306 stores information used by the processor 308 to fit the hearing prosthesis 102. In one example, the data storage 306 stores initial control settings, which are the control settings of the hearing prosthesis 102 prior to fitting. The data storage 306 may additionally store information usable by the processor 308 for modeling the hearing of the user and/or a model of the hearing prosthesis 102. The data storage 306 may also store computer programs executable by the processor 308, such as computer program that includes instructions for performing one or more steps of the methods 400, 500, 600, 700, and/or 800 described herein.

The processor 308 is configured to determine the control settings for the hearing prosthesis 102. In one example, the processor 308 accesses the data storage 306 to receive the initial control settings. In another example, the processor 308 receives initial control settings from the user via the user interface 304.

The processor 308 may also receive information from an additional device. In one example, the computing device 300 is connected to a database 330 through the external interface module 310. The processor 308 accesses the database 330 to identify information used for modeling the hearing of the user of the hearing prosthesis 102, such as the user's sensorineural hearing loss. The processor 308 also accesses the database 330 to retrieve a model for the hearing prosthesis 102 that is used in determining the control settings. In another example, the processor 308 identifies the information used for modeling the hearing of the user and/or the model of the hearing prosthesis 102 from the data storage 306.

The processor 308 is configured to receive a model output of the hearing prosthesis 102. The model output is an output of the model of the hearing prosthesis 102 based on the information used for modeling the user's hearing and the control settings for the hearing prosthesis 102.

In one example, the processor 308 communicates with a second computing device 332 via the external interface module 310. The processor 308 communicates with the second computing device 308 in order to receive the model output. For example, if the second computing device 332 is configured to model the hearing prosthesis 102, the processor 308 sends the initial control settings to the second computing device 332. The second computing device 332 models the sound received by the user of the hearing prosthesis and sends the model output of the model to the processor 308.

The external interface module 310 connects external devices, such as the hearing prosthesis 102, the database 330, and the second computing device 332, to the computing device 300. In one example, the external interface module 310 connects the computing device 300 to the external device via a wired connection interface. In another example, the interface module 310 connects the computing device 300 to the external device via a wireless connection interface. In yet another example, the interface module 310 includes one or more wired and/or wireless connection interfaces.

FIG. 4 is an example method 400 for determining control settings for a hearing prosthesis. A computing device may utilize the method 400 to determine control settings for hearing prosthesis. While the fitting system 100 is used for purposes of describing the method 400, it is understood that other devices may be used.

The method 400 and other method and processes disclosed herein may include one or more operations, functions, or actions as illustrated in the blocks. Although the blocks are illustrated in sequential order, these blocks may be performed in parallel and/or in a different order than those described herein. Also, the various blocks may be combined into fewer blocks, divided into additional blocks, and/or removed based upon the desired implementation.

In addition, for the method 400 and other processes and methods disclosed herein, the flow diagram shows functionality and operation of one possible implementation of one example. In this regard, each block may represent a module, a segment, or a portion of program code, which includes one or more instructions executable by a process for implementing specific logical functions or steps in the process. The program code may be stored on any type of computer readable medium, such as a storage device including a disk or hard drive, for example. The computer readable medium may include non-transitory computer readable media, such as a computer readable media that stores data for a short period of time, such as register memory, processor cache, or Random Access Memory (“RAM”). The computer readable medium may also include non-transitory computer readable media suitable as secondary or persistent long term storage, such as read-only memory (“ROM”), one time programmable memory (OTP), or the like. The computer readable medium may also include any other volatile or non-volatile storage systems. The computer readable medium may be considered computer readable storage medium, for example, or a tangible storage device.

In addition, for the method 400 and other processes and methods discussed herein, each block of FIG. 4 may represent circuitry that is wired to perform the specific logical functions of the process.

At block 402, the method 400 includes validating initial control settings and receiving a model output based on the initial control settings. The model output is an output of a model configured to simulate a user's perception of one or more sounds based on control settings for the hearing prosthesis 102. The model simulates a user's perception of a sound that is processed by the hearing prosthesis 102. The model is based on an operation profile of the hearing prosthesis 102, which includes operating characteristics of one or more components of the hearing prosthesis 102, such as the audio transducer 204, the sound processor 208, the stimulation component 212, and the implant unit 214. The model is also based on a hearing profile of the user of the hearing prosthesis 102. The hearing profile includes information indicative of the user's sensorineural hearing loss over one or more frequency ranges. Additionally, the model is based on a standard hearing profile, which includes information indicative of how a person with normal hearing perceives a sound. In one example, the model is based on more or fewer profiles that are suitable for use in simulating how the user of the hearing prosthesis 102 perceives a sound.

The model simulates the hearing prosthesis 102 receiving one or more sounds having frequencies that vary over one or more frequencies and outputs the model output. The model output includes an output characteristic for each of the one or more frequencies. In one example, the output characteristic is a loudness, a balance, a signal-to-noise ratio (SNR), a frequency response, an amount of feedback, an amount of distortion, or any other characteristic representative of how a user of the hearing prosthesis 102 perceives a sound. Additionally, the model output may include one or more additional output characteristics for each of the one or more frequency ranges.

In order ensure proper operation of the hearing prosthesis 102, the computing device 104 validates the initial control settings to ensure that the initial control specification is within an input specification. Validating the initial control settings is a prerequisite for performing additional steps of the method 400. The initial control settings include M settings for a parameter at M frequencies where M is an integer greater than one. Each of the M settings is compared to an input specification. In one example, the initial control settings are entered into an input component of the user interface module 304. If one of M settings is not within the input specification, the computing device 104 determines that control settings are invalid and does not use the control settings when running the model. The computing device 104 displays an error on an output component of the user interface module 304 indicating that the initial control settings are invalid and prompting the audiologist using the computing device 104 to enter valid initial control settings. In another example, the initial control settings are predetermined, validated values. The computing device 104 identifies the initial values by accessing the data storage 306 or the database 330.

In one example, the computing device 104 is configured to run the model using control settings for the hearing prosthesis 102. The computing device 104 receives the model output by running the model using the initial control settings. In another example, the computing device 104 receives the model output from the computing device 332. In this example, the computing device 104 sends the initial control settings to the second computing device 332, which is configured to run the model using control settings for the hearing prosthesis 102. The second computing device 332 runs the model using the initial controls sends and sends model output based on the initial control settings to the computing device 104, which the computing device 104 receives via the external interface module 310.

At block 404, the method 400 includes iteratively adjusting the initial control settings to achieve a model output having a target output characteristic at a target frequency. The target frequency represents a frequency that the audiologist designates to match as closely as possible to normal hearing. Because of design constraints of the hearing prosthesis 102 and the user's type and severity of hearing loss, the user of the hearing prosthesis 102 is not always able to perceive a sound as the person having normal hearing. That is, because of the complexity of the hearing prosthesis 102, the control settings for one or more frequencies may not have optimal control settings, thereby impairing the user's ability to perceive sounds at those frequencies.

Since it may not be possible to determine optimal control settings for each frequency, the target frequency represents a frequency at which the performance of the hearing prosthesis 102 is optimized. The computing device 104 determines the control settings for the hearing prosthesis 102 that result in a model output that has output characteristics at the target frequency that match normal human hearing as closely as possible. For instance, if the user considers the ability to hear human speech as a priority, the target frequency is set at 2 KHz, which is a frequency at which a majority of human speech occurs. The computing device 104 determines the control settings that will optimize the performance of the hearing prosthesis 102 at 2 KHz, thereby enhancing the user's ability to perceive human speech.

In one example, the computing device 104 iteratively determines the control settings that maximize the performance of the hearing prosthesis 102 at the target frequency. That is, the computing device 104 determines the control settings that allow the user of the hearing prosthesis 102 to perceive sound at the target frequency (e.g., a person speaking) as normally as possible (e.g., as a person without sensorineural hearing loss at the target frequency).

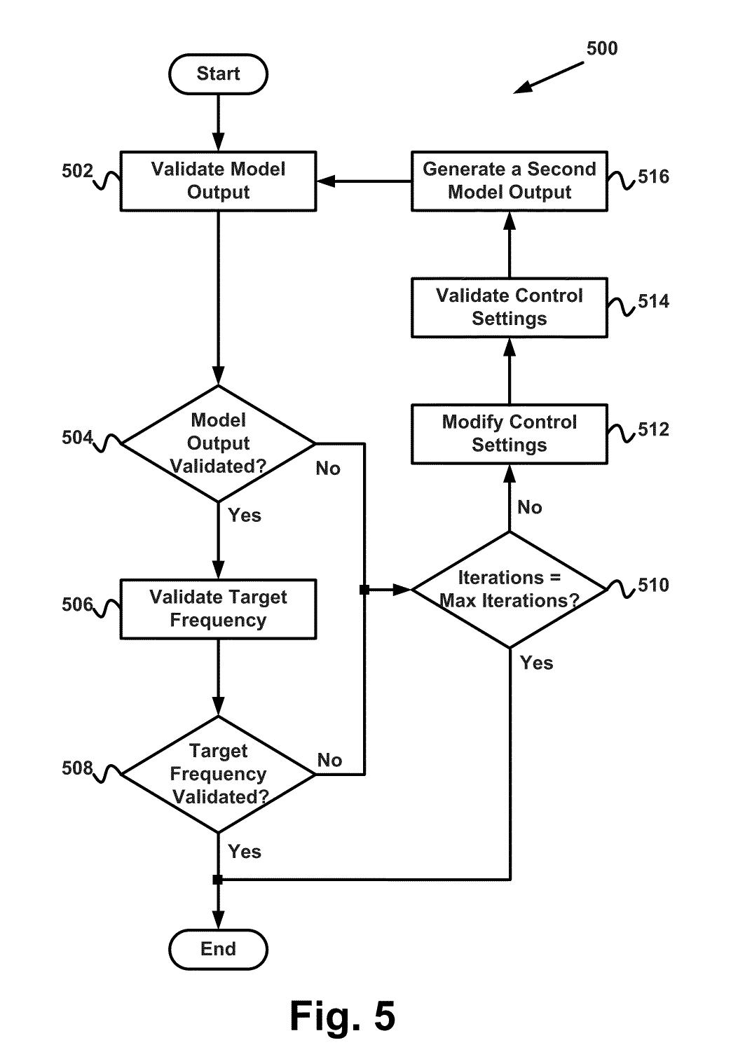

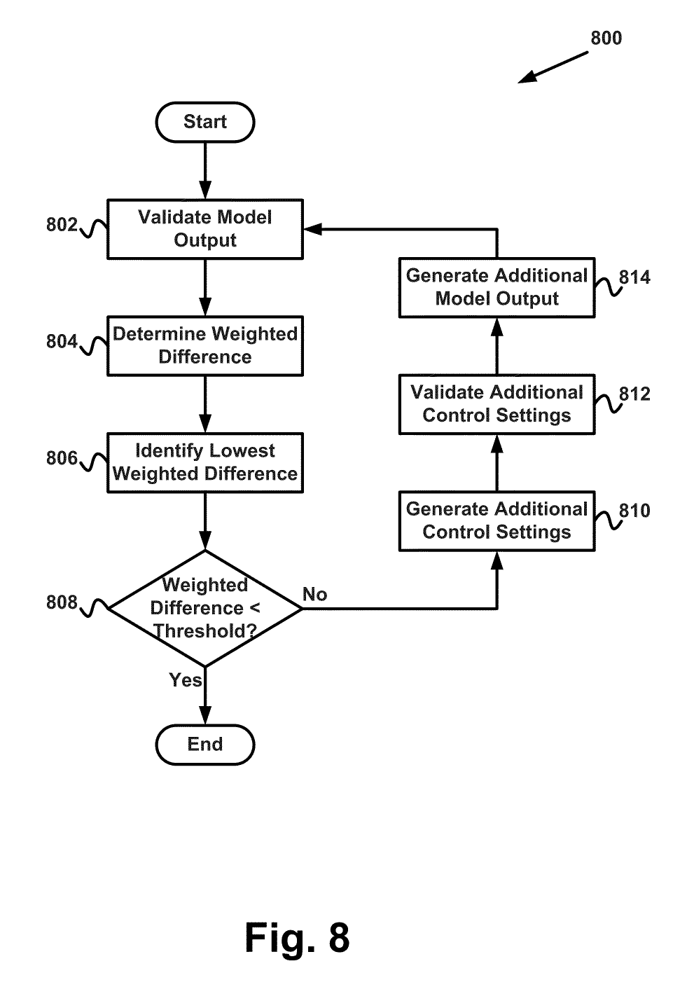

In another example, the computing device 104 is configured to iteratively adjust the control settings in order to achieve the model output having the target output characteristic at the target frequency. For example, if the target output characteristic is a target gain, the computing device is configured to iteratively adjust the control settings in order to achieve the model output having the target gain at the target frequency. To determine the control settings that result in the model output having the target output characteristic, the computing device 104 may employ either of the methods described in FIGS. 5 and 8.

At block 406, the method 400 includes sending a signal to the hearing prosthesis that includes information indicative of the control settings. Once the computing device 104 has determined the control settings that result in the model output having the target output characteristic at the target frequency, the computing device 104 sends the signal indicative of the control settings to the hearing prosthesis 104 via the link 110. Alternatively, the computing device 104 includes information indicative of last control settings generated during the iterative process performed in step 404. For instance, if the computing device 104 performed a maximum number of iterations of an iterative process, the computing device 104 includes the last control settings generated during the iterative process in the signal sent to the hearing prosthesis 102.

FIG. 5 is a flow diagram of a method 500 depicting a first example of an iterative process for determining the control settings at block 404 of the method 400.

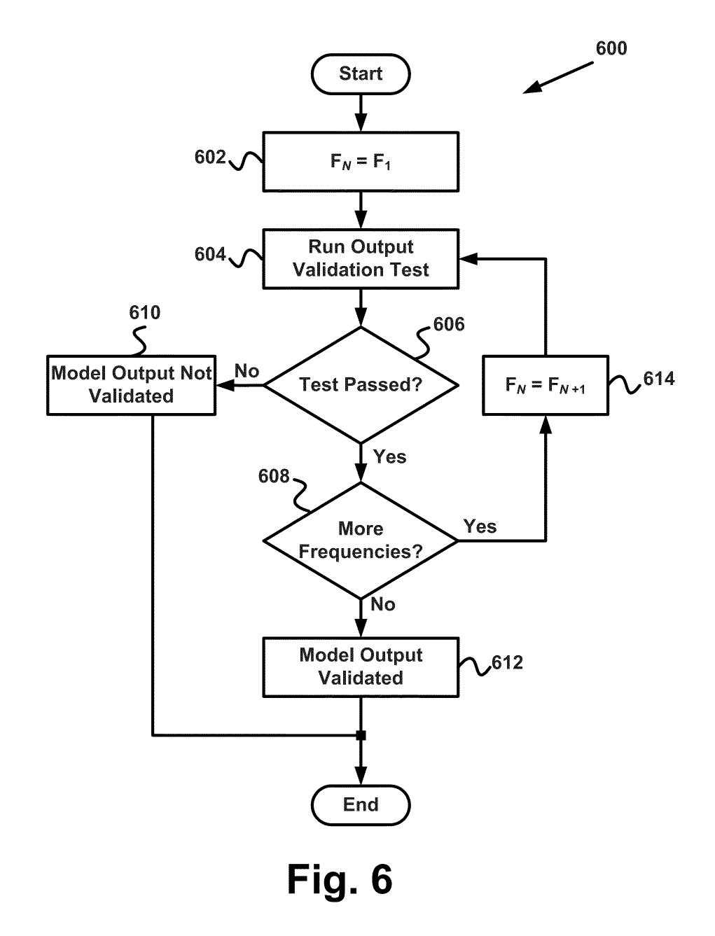

At block 502, the method 500 includes validating the frequencies of the model output. To prevent damage to the hearing prosthesis 102 and/or injury to the user of the hearing prosthesis 102, the computing device 104 determines whether one or more output characteristics of the model output at one or more frequencies are within one or more specifications. One example method for validating the model output frequency is described with respect to FIG. 6.

At block 504, the method 500 includes determining whether the model output is validated. If the computing device 104 determined that the model output was validated at block 502, then the computing device 104 validates the target frequency, at block 508. If the computing device 104 determined that the model output was not validated at block 502, the computing device determines whether additional iterations of the method 500 are performed, at block 506.

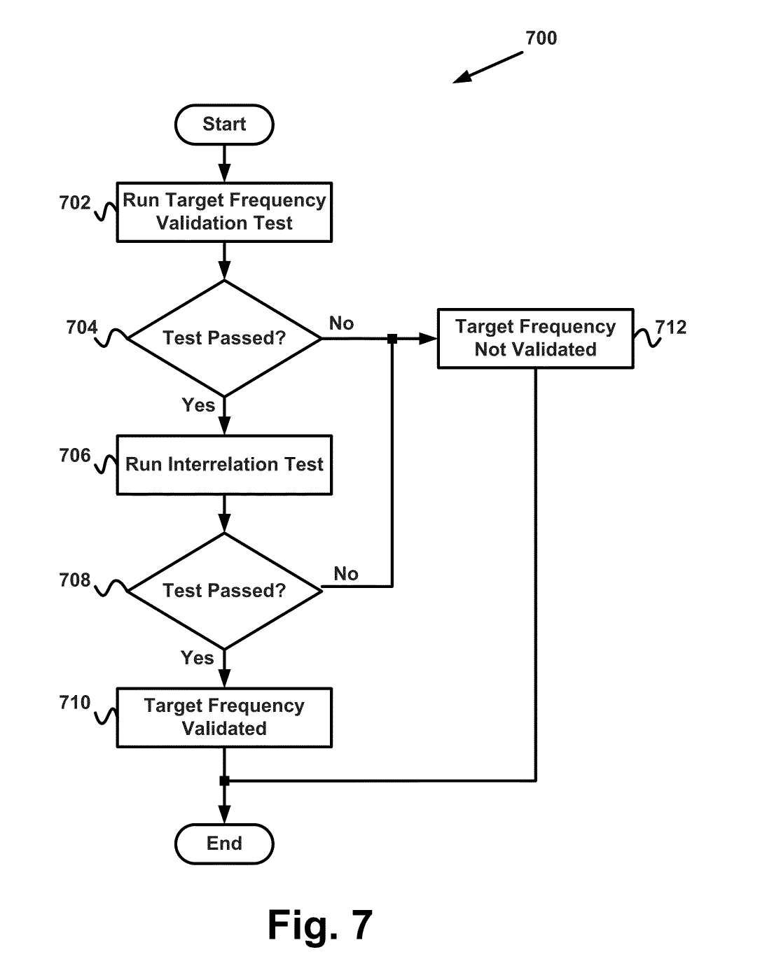

At block 506, the method 500 includes validating the target frequency. The computing device 104 validates the target frequency by comparing the target frequency to one or more specifications. One example method for validating the target frequency is described with respect to FIG. 7.

At block 508, the method 500 includes determining whether the target frequency is validated. If the computing device 104 determined that the target frequency was validated at block 506, then the method 500 ends. If the computing device 104 determined that the target frequency was not validated at block 510, the computing device 104 determines whether additional iterations of the method 500 are performed, at block 510.

At block 510, the method 500 includes determining whether the number of iterations of the method 500 equals the maximum number of iterations. Due to the complexity of human hearing, the computing device 104 may perform a number of iterations of the method 500 before determining the control settings that result in both a validated model output and a validated target frequency. To reduce the number of iterations the computing device 104 performs and the amount of time taken to fit the hearing prosthesis 102 to the user, the computing device 104 performs a maximum number of iterations. The maximum number of iterations is a number of iterations of the method 500 determined prior to fitting. In one example, the audiologist can adjust the maximum number of iterations.

The computing device 104 stores the number of iterations of the method 500 performed and the maximum number of iterations in the data storage 306. In one example, the maximum number of iterations is about one hundred iterations. In another example, the maximum number of iterations is any number of iterations suitable for fitting the hearing prosthesis 102 to the user. Additionally, the maximum numbers of iteration may depend on the input 120 received from the audiologist using the computing device 104 to fit the hearing prosthesis 102 to the user.

If the computing device 104 determines that the number of iterations of the method 500 is equal to the maximum number of iterations, then the method 500 ends. If computing device 104 determines that the number of iterations is not equal to the maximum number of iterations, the computing device 104 modifies the control settings, at block 512.

At block 512, the method 500 includes modifying the control settings. The computing device 104 is configured to modify the control settings based on the target frequency. That is, the computing device 104 modifies the control settings to achieve a model output having a specific output characteristic at the target frequency.

How the computing device modifies the one or more subsets of parameter settings depends on whether the model output was not validated at block 502 or whether the target frequency was not validated at block 506. If the model output was not validated, the computing device 104 is configured to make a first modification to the control parameter based on the frequency that did not pass the output validation test. If the target frequency was not validated, the computing device 104 is configured to modify the control settings based on the reason the target frequency was not validated. For instance, if the target frequency was not validated because the model output did not pass the target frequency validation test, the computing device 104 is configured to modify the control settings in order to achieve a model output that passes the target frequency validation test. Alternatively, if the model output did not pass the frequency interrelation test, the computing device 104 is configured to modify the control settings in order to achieve a model output that passes the frequency interrelation test.

Because of the complexity of models for human hearing, making a modification to one subset of parameter settings may impact the response of the hearing prosthesis 102 at other frequencies. As a result, the computing device 104 may make one or more additional adjustments to one or more additional subsets of parameter settings based on the first modification.

At block 514, the method 500 includes validating the control settings. The computing device 104 validates the control settings generated at block 512 using the same or a substantially similar process as the validation process described with respect to block 402 of the method 400. The control settings are used to generate a second model output, at block 516.

Once the computing device 104 generates the second model output, the computing device 104 continues returns to block 502 to validate the second model output. The computing device 104 continues performing the steps of blocks 502-514 until either (i) both the model output and the target frequency are validated, or (ii) the computing device 104 performs the maximum number of iterations of the method 500

In the above description of the method 500, the computing device 104 determined the control settings for the hearing prosthesis 102 based on a single target frequency. In another example, the computing device 104 employs the method 500 to determine control settings for the hearing prosthesis 102 using multiple target frequencies. In this example, the method 500 includes performing the steps of block 510 for each target frequency. The computing device 104 is also configured to modify one or more subsets of parameters settings in order to maximize the correlation between the output target characteristics and the reference target characteristics.

FIG. 6 is a flow diagram that depicts a method 600 represents an example method for validating a model output. The method 600 is one example of a method for validating the model output at block 502 of the method 500. While the fitting system 100 and computing device 300 are used for purposes of describing the method 400, it is understood that other devices may be used.

At block 602, the method 600 includes setting a test frequency F_{N} equal to a first frequency F₁, where N is an integer greater than or equal to one. The model output includes at least one output characteristics at M frequencies, where M is an integer greater than or equal to 1. In one example, the M frequencies range from about 250 Hz to about 8 KHz. In this example, the first frequency F₁ is about 250 Hz.

At block 604, the method 600 includes running an output validation test. In one example, the output validation test includes determining whether a first output characteristic for the model output at the test frequency F_{N} is within a first specification. The first specification represents a maximum and/or a minimum allowable value for the first output characteristic based on operational constraint of the hardware and/or software of the hearing prosthesis 102. For example, if the first output is a gain, the first specification is a maximum gain. Alternatively, if the first output characteristic is a compression ratio, the first specification includes both a minimum compression ratio and a maximum compression ratio. In an alternative example, the output validation test includes determining whether one or more output characteristics for the model output at the test frequency F_{N} are within one or more specifications.

The model output passes the output validation test at the test frequency F_{N} if the first output characteristic is within the first specification. In one example, the first output characteristic is within the first specification if the first output characteristic is less than or equal to the first specification. In another example, the first output characteristic is within the first specification if the first output characteristic is less than the first specification. In yet another example, the first output characteristic is compared to the specification using any relational operator or combination of relational operators suitable for determining whether the first output characteristic is within the specification.

At block 606, the method 600 includes determining whether the model output passes the output validation test at the test frequency. If the model output passed the output validation test, the computing device 104 determines that test frequency is validated and determines whether there are additional frequencies to validate, at block 608. If the model output did not pass the output validation test, the computing device 104 determines that the model output is not validated, and the method 600 ends.

At block 608, the method 600 includes determining whether there are more frequencies to validate. If N equals M, then the first output characteristic for each of the M frequencies of the model output has passed the output validation test. The computing device 104 determines that model output is validated, at block 612, and the method 600 ends.

If N does not equal M, then there are additional frequencies of the model output to validate. The test frequency F_{N} is changed to the next test frequency F_{N+1}, at block 614. After perform in the steps of block 614, the method 600 returns to block 604 to determine whether the N^{th} output characteristic of the model output is within the N^{th} specification. The computing device 104 continues performing iterations of the method 600 until the computing device determines that model output is either validated or not validated for all test frequencies.

FIG. 7 is a flow diagram of a method 700. The method 700 represents an example method for validating a target frequency of a model output.

At block 702, the method 700 includes running a target frequency validation test. The target frequency validation test includes comparing an output target characteristic to a reference target characteristic. The output target characteristic is an output characteristic of the model output at the target frequency. For example, if the target frequency is 2 KHz, the output target characteristic is a gain of the model output at 2 KHz, a compression ratio of the model output at 2 KHz, a maximum power output at 2 kHz, a SNR of the model output at 2 KHz, or the value of any other characteristic of the model output at 2 KHz.

The reference target characteristic is a reference characteristic of a reference output at the target frequency. The reference output represents a perception by a person having normal hearing of the one or more sounds used by the model to generate the model output. The reference output includes a reference characteristic at one or more frequencies. The reference characteristic at a given frequency represents how the person having normal hearing perceives the characteristic of the one or more sounds at the given frequency. In one example, the reference characteristic is a loudness, a balance, a SNR, a frequency response, an amount of feedback, an amount of distortion, or any other characteristic representative of how a person with normal hearing perceives a sound.

The reference target characteristic is the reference characteristic of the reference output at the target frequency. In one example, the computing device 104 identifies the reference target characteristic by accessing the data storage 306. In another example, the computing device 104 receives the reference output from an external device, such as the database 330 or the second computing device 332.

In one example, comparing the output target characteristic to the reference target characteristic includes determining a difference between the output target characteristic and the reference target characteristic. For example, if the output target characteristic is an output SNR at the target frequency and the reference characteristic is a reference SNR at the target frequency, the comparison is the difference between the output SNR and the reference SNR. In another example, the comparison includes any comparative technique suitable for use in fitting the hearing prosthesis 102 to the user.

If the difference between the output target characteristic and the reference target characteristic is within a tolerance, the computing device 104 determines that the target frequency is validated. If the difference between the output target characteristic and the reference target characteristic is not within a tolerance, the computing device 104 determines that the target frequency is not validated. In one example, the difference is within the tolerance if the distance is less than or equal to the tolerance. In another example, the difference is within the tolerance if the difference is less than the tolerance. In yet another example, the difference is compared to the tolerance using any relational operator or combination of relational operators suitable for determining whether the first output characteristic is within the specification.

At block 704, the method 700 includes determining whether the model output passed the target frequency validation test. If the model output passed the target frequency validation test, the computing device 104 runs a frequency interrelation test, at block 706. If the model output did not pass the target frequency validation test, the computing device 104 determines that the target frequency is not validated at block 712, and the method 700 ends.

At block 706, the method 700 includes running a frequency interrelation test. The frequency interrelation test includes determining an interrelation difference between a first output characteristic of the model output at a first frequency and a second output characteristic of the model output at a second frequency. The first frequency and the second frequency are any two frequencies of the model output. For instance, if the output characteristic is the compression ration, the interrelation difference is the difference between a first compression ration at the first frequency and a second compression ratio at the second frequency.

The computing device 104 performs the interrelation test for every combination of frequency pairs. For instance, if the model output includes output characteristics at three frequencies, the computing device 104 determines the following interrelation differences: (i) a first interrelation difference between a first output characteristic at the first frequency and second output characteristic at the second frequency, (ii) a second interrelation difference between the first output characteristic and a third output characteristic at a third frequency, and (iii) a third interrelation difference between the second output characteristic and the third output characteristic.

The computing device 104 determines whether the interrelation difference between any two frequencies is within an interrelation specification. In one example, the interrelation difference is within the interrelation specification if the interrelation difference is less than or equal to the interrelation specification. In another example, the interrelation difference is within the interrelation specification if the interrelation difference is less than the interrelation specification. In yet another example, the interrelation difference is compared to the interrelation specification using any relational operator or combination of relational operators suitable for determining whether the interrelation difference is within the interrelation specification.

If the computing device 104 determines that the interrelation difference is within the interrelation specification for the frequencies included in the model output, the computing device 104 determines that the model output passes the frequency interrelation test. Otherwise, the computing device 104 determines that the model output did not pass the frequency interrelation test.

At block 708, the method 700 includes determining whether the model output passed the frequency interrelation test. If the model output passed the frequency interrelation test, the computing device 104 determines that the target frequency is validated at block 710, and the method 700 ends. If the model output did not pass the frequency interrelation test, the computing device determines that the target frequency is not validated at block 712, and the method 700 ends.

FIG. 8 is a flow diagram of a method 800 that depicts a second example of an iterative process for determining the control settings at block 404 of the method 400.

At block 802, the method 800 includes validating the model output. The computing device 104 is configured to validate the model output, perhaps by employing the method 600 described with respect to FIG. 6. In one example, the computing device 104 validates multiple model outputs. In this example, a first set of model outputs that are validated, and a second set of model output is not validated. The computing device 104 retains the first set of model outputs and uses the first set of model outputs when performing subsequent steps of the method 800. The computing device 104 does not retain the second set of model outputs and does not use the second set of model outputs when performing subsequent steps of the method 800.

At block 804, the method 800 includes determining a weighted difference between a predicted output and a reference output. In one example, the weighted difference includes determining a sum of one or more differences between a predicted output characteristic and a reference output characteristic at one or more frequencies. The weight of each of the one or more differences depends on the frequency corresponding to each of the one or more differences. In another example, the weighted difference is determined using any mathematical or statistical operation or combination of operations suitable for determining the weighted difference between the predicted output and the reference output

In one example, the computing device 104 determines the weighted difference of the model output by determining a weighted sum of square errors given by the following equation:

WD=Σᵢ₌^{N}Σₖ₌₁^{M}Wₖᵢ(Xₖ(fᵢ)−Rₖ(fᵢ))²

where WD is the weighted difference for the model output, N is the number of frequencies of the model output, M is the number of output characteristics for each frequency, Wₖᵢ is the weighting factor for k^{th} output characteristic at the i^{th} frequency, Xₖ(fᵢ) is the k^{th} output characteristic of the model output at the i^{th} frequency, and Rₖ(fᵢ) is the k^{th} reference characteristic of the reference output at the i^{th} frequency. In another example, the weighted difference is determined using any process, method, or algorithm now known or later discovered that is suitable for use in fitting a hearing prosthesis.

The computing device 104 identifies the values of the weighting factor by accessing a data storage, such as the data storage 306 or the database 330. Weighing square differences between output target characteristics and reference target characteristics results in the weighted error being dependent on the square difference having a greater weighting factor. In one example, weighting factors have a value that is an integer greater than zero. In another example, the weighting factor is any real number between zero and one.

The weighting factor for the target frequency has the greatest value. Therefore, the greater the difference between an output target characteristic and a reference target characteristic, the greater the weighted error for the model output. Or in other words, the closer the output target characteristic is to the reference target characteristic, the smaller the weighted error is. In one example, the computing device 104 is configured to determine the weighted error using multiple target frequencies. In this example, the weighting factor for each target frequency has the same value.

The weighting factors for the remaining N frequencies may depend on any number of factors. In one example, the weighting factor for the i^{th} frequency depends on i^{th} frequency's proximity to the target frequency; the closer the i^{th} frequency is to the target frequency, the greater the weighting factor for the i^{th} frequency. In another example, the audiologist using the computing device 104 to fit the hearing prosthesis 102 to the user determines the weighting factor for the i^{th} frequency based on the user's type and severity of hearing loss. In still another example, the weighting factor for the i^{th} frequency is determined using any method or process now known or later discovered suitable for fitting a hearing prosthesis.

At block 806, the method 800 includes identifying the model output with a lowest weighted difference. In an example in which there is one model output, the computing device 104 may omit the steps of block 806. In an example in which includes multiple model outputs having multiple weighted difference, the computing device 104 identifies the lowest weighted difference from the multiple weighted differences.

To illustrate the steps of block 804 and 806, consider the following example in which the computing device 104 identifies the lower of two weighted differences. At block 804, the computing device determines a weighted difference of a first model output and a second model output. The first model output includes a first output characteristic at a first frequency and a second output characteristic at a second frequency. The second model output includes a third output characteristic at the first frequency and a fourth output characteristic at the second frequency. The reference output, in this example, includes a first reference characteristic at the first frequency and a second reference characteristic at the second frequency. For illustrative purposes, the first frequency is the target frequency.

In this example, the computing device 104 determines the weighted difference using a sum of square errors technique. To determine a first weighted difference WD₁ between the first model output and the second model output, the computing device 104 determines a first square difference between the first output characteristic X₁ and a first reference characteristic R₁ and a second square difference between the second output characteristic X₂ and a second reference characteristic R₂. The square first difference is then multiplied by a first weighting factor W₁ to get a first product, and the second square difference is multiplied by a second weighting factor W₂ to get a second product. Because the first frequency is the target frequency, the first weighting factor W₁ is greater than the second weighting factor W₂. The first product is added to the second product to get the first weighted difference WD_{I}.

To determine a second weighted difference WD₂ between the second model output and the second model output, the computing device 104 determines a third square difference between the third output characteristic X₃ and the first reference characteristic R₁, and a fourth square difference between the fourth output characteristic X₄ and the second reference characteristic R₂. The third difference is then multiplied by the first weighting factor W₁ to get a third product, and the fourth difference is multiplied by the second weighting factor W₂ to get a fourth product. The third product is added to the fourth product to get the second weighted difference WD₂. The following equations represent the first weighted difference and the second weighted difference:

WD₁=W₁(X₁−R₁)²+W₂(X₂−R₂)²

WD₂=W₁(X₃−R₁)²+W₂(X₄−R₂)²

The computing device 104 determines whether the first weighted difference is less than the second weighted difference. If the first weighted difference is less than second weighted difference, then the computing device 104 determines whether the first weighted difference is less than the threshold difference, at block 808. The computing device 104 also discards the second model output. If the first weighted difference is not less than the second weighted difference, then the computing device 104 determines whether the second weighted difference is less than the threshold, at block 808, and discards the first model output.

At block 808, the method 800 includes determining whether the lowest weighted difference is less than a threshold difference. If the lowest weighted difference is less than the threshold difference, then the method 800 ends. If the computing device 104 determines that the lowest weighted difference is greater than or equal to threshold error, the computing device 104 generates additional control settings, at block 810.

In another example, the computing device 104 determines the control settings are the optimal control settings if the lowest weighted difference is less than or equal to the threshold difference. In this example, the method 800 ends if the computing device 104 determines that lowest weighted difference is less than or equal to the threshold difference. Otherwise, the computing device 104 proceeds to block 810 to determine the additional control settings.

In yet another example, the computing device 104 determines, at block 808, whether a change in the weighted difference is less than a threshold value. Due to the complexity of human hearing, the computing device 104 may not be able to determine the control settings that result in a model output having a weighted difference that is less than the threshold difference. If the computing device 104 determines that difference in a number of least weighted errors is about constant, the computing device 104 determines that control settings corresponding to a last least weighted error represents a best solution based on the characteristics of the hearing prosthesis 102 and the user's hearing loss, and the method 800 ends.

At block 810, the method 800 includes generating additional control settings. The additional control settings are based on the control settings corresponding to the model output having the lowest weighted difference, as determined at block 806, for example. To ensure that the additional control settings include at least one set of valid control settings, the additional control settings include the control settings corresponding to the model output having the lowest weighted difference.

In one example, the computing device 104 is configured to generate L permutations of additional control settings, where L is an integer greater than zero. Each of the L permutations of additional control settings includes N parameter settings for M frequencies, where M and N are also integers greater than one. The computing device 104 may generate the permutations in any number of possible ways. For instance, an adjustment is made to increase and decrease each of the N parameter settings by a scalar value, resulting in total number of permutations equaling (M×N×2)-permutations of the L additional control signals. Alternatively, the adjustment is made to a setting for each of the M frequencies, resulting in M permutations of the L additional control settings. As an additional example, the adjustment is made to a setting for each of the N control parameters, resulting in N permutations of L additional control settings. In yet a further example, each of the preceding examples is summed to generate a total of (M×N×2+M+N) permutations of the L additional control settings.

In another example, the computing device 104 is configured to determine the additional control settings based on the weighted differences for one or more frequencies that were determined at block 804. In yet another example, the computing device 104 generates the additional control settings using any process, method, or algorithm suitable for determining control settings for a hearing prosthesis.

At block 812, the method 800 includes validating the additional control settings. The computing device 104 validates the additional control settings generated at block 810 using the same or a substantially similar process as for validating the initial control settings described with respect to block 402 of the method 400. The additional control settings are used to generate additional model outputs, at block 814. In an example in which the computing device 104 generates the L permutations of additional control settings, additional model outputs corresponding to each of the L permutations are generated at block 814.

Once the computing device 104 generates the additional model output, the method 800 returns to block 802 to validate the additional model output. The computing device continues performing iterations of the steps of blocks 804-816 until the lowest weighted error is less than, or in some example less than or equal to, the threshold difference. In one example, the computing device 104 performs a certain number of iterations of the steps of blocks 804-816. For instance, if a maximum number of iterations is one hundred, the computing device 104 performs one hundred iterations of the steps of block 804-816 before the method 800 ends.

In another example, the computing device 104 determines the weighted difference for the predicted outputs for each of the L permutations of control settings passing the validation tests, and sends the control settings corresponding to the lowest weighted difference to the hearing prosthesis. Alternatively, if the computing device 104 determines that the lowest weighted difference is greater than a previous lowest weighted difference, the computing device 104 sends the control settings corresponding to the predicted output having the previous lowest weighted difference to the hearing prosthesis.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method comprising:
receiving, by a computing device configured to fit a hearing prosthesis to a user of the hearing prosthesis, a model output of the hearing prosthesis based on control settings usable by the hearing prosthesis to process a sound;
determining if the model output passes a validation test, wherein the validation test includes determining that a difference between the model output and a reference output at a target frequency is within a specification, wherein the reference output that is based on normal human hearing at the target frequency; and
in response to determining that the model output passes the validation test, sending a signal to the hearing prosthesis that contains information indicative of the control settings.

2. The method of claim 1, wherein determining that the difference is within the specification includes one of:
(i) determining that the difference is less than the specification; and
(ii) determining that the difference is less than or equal to the specification.

3. The method of claim 1, wherein the control settings include information indicative of one or more settings of one or more parameters used by the hearing prosthesis to process the sound.

4. The method of claim 1, further comprising, in response to determining that the model output failed the validation test, determining a second control settings based on the difference.

5. The method of claim 4, further comprising:
receiving a second model output that is based on the second control settings;
determining a second difference between the second model output and the reference output;
determining whether the second difference is within the specification; and
in response to determining that the second difference is within the specification, sending a second signal to the hearing prosthesis that contains information indicative of the control settings.

6. The method of claim 1, wherein the model output includes a first output characteristic at a first frequency and a second model output characteristic at a second frequency.

7. The method of claim 6, wherein the validation test further includes:
determining that a frequency interrelation difference is within of a frequency interrelation specification, wherein the frequency interrelation difference is a difference between the first output characteristic and the second output characteristic.

8. A method comprising:
receiving, by a computing device configurable to determine control settings for a hearing prosthesis without causing the hearing prosthesis to deliver a stimulus to a user, a first model output of a model that simulates how the user perceives a sound when using the hearing prosthesis with a first set of control settings, a second model output of the model that simulates how the user perceives the sound when using the hearing prosthesis with a second set of control settings, and a reference output based on normal human hearing, wherein the first set of control settings and the second set of control settings are usable by the hearing prosthesis to process the sound;
determining a first weighted difference between the first model output and the reference output that includes giving more weight to a first difference between the first model output and the reference output at a first frequency than to a second difference between the first model output and the reference output at a second frequency;
determining a second weighted difference between the second model output and the reference output that includes giving more weight to a third difference between the second model output and the reference output at the first frequency than to a fourth difference between the second model output and the reference output at the second frequency;
determining, by the computing device, whether the first weighted difference is less than the second weighted difference;
responsive to determining that the first weighted difference is less than the second weighted difference, determining, by the computing device, whether the first weighted difference is within a tolerance; and
responsive to determining that the first weighted difference is within the tolerance, sending to the hearing prosthesis a signal that includes information indicative of the first set of control settings.

9. The method of claim 8, wherein:
the first model output includes a first output characteristic at the first frequency and a second output characteristic at the second frequency;
the second model output includes a third output characteristic at the first frequency and a fourth output characteristic at the second frequency; and
the reference output includes a first reference characteristic at the first frequency and a second reference characteristic at the second frequency.

10. The method of claim 9, wherein:
the first difference is a first square difference between the first output characteristic and the first reference characteristic;
the second difference is a second square difference between the second output characteristic and the second reference characteristic;
the third difference is a third square difference between the third output characteristic and the first reference characteristic; and
the fourth difference is a fourth square difference between the fourth output characteristic and the second reference characteristic.

11. The method of claim 8, wherein:
determining the first weighted difference includes multiplying the first difference by a first factor and multiplying the second difference by a second factor, wherein the first factor and the second factor are greater than zero, and wherein the first factor is greater than the second factor; and
determining the second difference includes multiplying the third difference by the first factor and multiplying the fourth difference by the second factor.

12. The method of claim 9, wherein the first output characteristic, the second output characteristic, the third output characteristic, the fourth output characteristic, the first reference characteristic, and the second reference characteristic are each one of a gain, a compression ratio, and a maximum power output, loudness, a balance, a signal-to-noise ratio, or a frequency response.

13. The method of claim 8, wherein the first weighted difference and the second weighted difference are based on sums of square errors.

14. The method of claim 8, wherein, responsive to determining that the first weighted difference is outside of the tolerance, the method further includes:
generating N permutations of additional sets of control settings, wherein N is an integer greater than or equal to one;
sending the N permutations of additional sets of control settings to a computing device configured to run the model using each of the N permutations of additional control settings;
receiving N additional model outputs from the computing device, wherein each of the N additional model outputs is generated from one of the N permutations of control settings;
determining N weighted differences, wherein each of the N weighted differences is a weighted difference between one of the N additional model outputs and the reference output;
identifying from the N additional model outputs a third model output having a lowest weighted difference of the N weighted differences;
determining whether the lowest weighted difference is within the tolerance; and
in response to determining that the lowest weighted difference is within the tolerance, sending a second signal to the hearing prosthesis that includes information indicative of a third set control settings, wherein using the third set of control settings.

15. The method of claim 14, wherein the N permutations of additional sets of control settings include the first set of control settings.

16. A device comprising:
an interface component configured to send one or more control settings to a hearing prosthesis, wherein the hearing prosthesis uses the one or more control settings to process a sound; and
a processor configured to:
receive from a model output based on the initial control settings;
determine whether the first model output is outside of a specification by determining whether a first output characteristic of the first model output at a target frequency exceeds a threshold value; and
in response to determining that the first output characteristic is outside of the specifications,
generate second control settings that result in the second predicted output having a second output characteristic that is within the specification; and
send a signal to the hearing prosthesis via the interface component that includes the second control settings.

17. The device of claim 16, wherein the threshold value is based on a reference output characteristic at the target frequency, wherein the reference output characteristic is based on normal human hearing at the target frequency.

18. The device of claim 16, wherein the threshold value is based on an operational constraint of the hearing prosthesis.

19. The device of claim 16, wherein the processor is further configured to:
in response to determining that the first output characteristic is within the specification, send a second signal to the hearing prosthesis via the interface component that includes information indicative of the initial control settings.

20. The device of claim 16, wherein the interface component includes a user interface configured to receive an input from a user of the computing device, wherein the processor is further configured to receive the initial control settings from the user interface.
